(19) United States
(12) Patent Application Publication
(10) Pub. No.: US 2015/0342773 A1
(43) Pub. Date: Dec. 3, 2015

(54) TOES AND FINGERS CAP FOR USE WITH A CAST OR SPLINT

(71) Applicant: Alanna Wargula, Lakeport, CA (US)

(72) Inventor: Alanna Wargula, Lakeport, CA (US)

(21) Appl. No.: 14/295,304

(22) Filed: Jun. 3, 2014

(51) Int. Cl. A61F 5/01

(52) U.S. Cl. CPC A61F 5/01

(57) ABSTRACT

A cast and splint finger and toe cap is disclosed. The finger and toe cap is intended for combination with a splint, plaster or fiberglass cast of the type which is applied to the hand or foot of a fracture patient leaving the fingers or toes uncovered and protected. The cap comprises two elongated linear members each defining a plurality of flexible hinges and ridge protrusions along their length wherein the members are connected together for rotation. In use, these members are rotated and partially bent using the hinges for attachment using the ridge protrusions to opposite sides at the end of the cast whereby the cap protects a patient's digital extremities during orthopedic healing by forming a protective cage.

### TECHNICAL FIELD

The present invention relates to an orthopedic cap for use in combination with an orthopedic cast attached on either the foot or hand for protecting either the toes or fingers of a patient wearing the orthopedic cast.

### BACKGROUND ART

For fractures and sprains of the hand and foot, the usual procedure is to immobilize the hand or foot by applying a splint, plaster or fiberglass cast. It is well known in the prior art to partially cover the hand or foot by leaving the cast open such that the fingers and toes may be flexed and exercised, thereby facilitating healing by improving blood circulation. However, this practice leaves the fingers and toes exposed and vulnerable to accidental injury. For example, ambulatory patients, and particularly young active children, often bump their exposed fingers and toes into and on floors, walls, doors and furniture.

Furthermore, once placed in a splint or cast, bed clothing exerts uncomfortable pressure on the fingers and toes since they cannot be rotated. This problem is sometimes dealt with by constructing the cast with a plaster extension built out beneath the fingers and toes. However, this solution is not satisfactory because it is expensive and time consuming to construct and often prevents downward flexion of the fingers and toes, thereby limiting the extent of finger and toe exercise.

Currently, in the prior art there are a number of cast protective devices. One such protective device is disclosed in U.S. Pat. No. 3,832,997 issued Sep. 3, 1974 to Cappelletti, which teaches a U-shaped member adapted to be attached at the ends thereof to the opposite sides of a plaster cast and extending around the front of the cast to protect a patient's toes, and additionally serves as a toe traction splint when required. However, this device leaves the top and bottom of a patient's toes exposed and therefore unprotected leaving the toes vulnerable to injury.

Accordingly, there exists a need for an orthopedic cap for use with either plaster or fiberglass casts that will keep the toes and fingers of a patient protected by surrounding these appendages on all sides. The present invention solves this problem and more in a unique and novel fashion.

### SUMMARY OF THE INVENTION

A cast and splint finger and toe cap is disclosed. The finger and toe cap is intended for combination with a splint, plaster or fiberglass cast of the type which is applied to the hand or foot of a fracture patient leaving the fingers or toes uncovered and protected. The cap comprises two elongated linear members each defining a plurality of flexible hinges and ridge protrusions along their length wherein the members are connected together for rotation. In use, these members are rotated and partially bent using the hinges for attachment using the ridge protrusions to opposite sides at the end of the cast whereby the cap protects a patient's digital extremities during orthopedic healing by forming a protective cage.

In accordance with these and other objects, which will become apparent hereinafter, the instant invention will now be described with particular reference to the accompanying drawings.

### DESCRIPTION OF THE DRAWINGS

In the accompanying drawings:

FIG. 1 is a front view of the cap in accordance with a preferred embodiment of the present invention;

FIG. 2 is the same front view of the cap of FIG. 1 in a bent configuration;

FIG. 3 is a perspective view of the cap of FIG. 1 in another bent configuration;

FIG. 4 is a top view of the cap of FIG. 1 in still yet another bent configuration;

FIGS. 5 through 7 are perspective, side and top views, respectfully illustrating the use of the cap shown in FIG. 1 in combination with a plaster cast covering a patient's foot in accordance with one embodiment of the present invention; and

FIGS. 8 and 9 are top and side views, respectfully illustrating the use of the cap shown in FIG. 1 in combination with a plaster cast covering a patient's hand in accordance with one embodiment of the present invention.

### DESCRIPTION OF EMBODIMENTS

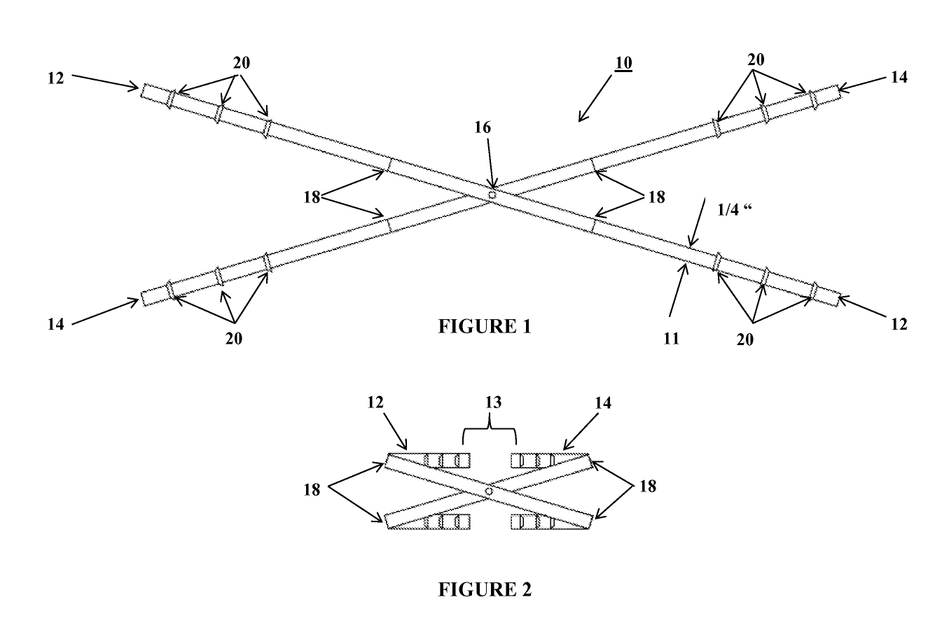

Referring now to FIGS. 1 through 4 there is shown a toes and fingers cap 10 used to protect the fingers and toes from stubbing/catching/etc., when wearing a cast during podiatric/orthopedic healing. The toes and fingers cap 10 may be used with fiberglass/plaster casts or splints. Turning once again to FIGS. 1 through 4, the toes and fingers cap 10 comprises a first generally elongated linear member 12 defining a plurality of flexible hinges 18 and ridge protrusions 20. Identical in shape and length to first member 12 is a second member 14 which also defines an identical plurality of flexible hinges 18 and ridge protrusions 20.

The first and second member 12 and 14 are connected together at a center point 16 which in one embodiment may be by use of a rivet thereby allowing the first and second member 12 and 14 to fully rotate and swivel around each other. As shown in FIG. 1, the ridge protrusions 20 are located at opposite ends at an equal distance near a distal end from the center point 16 of each member 12 and 14 and are used for cast attachment as will be more fully described below. Turning once again to FIG. 1, pair of flexible hinges 18 are located an equal distance between the center point 16 and ridge protrusions 20 of each member 12 and 14 and are also used for cast attachment as will be more fully described below. As shown in FIG. 2 the length of the first and second member 12 and 14 when fully bent and folded define a space 13 wherein the first and second member may by way of example only be a quarter inch in diameter.

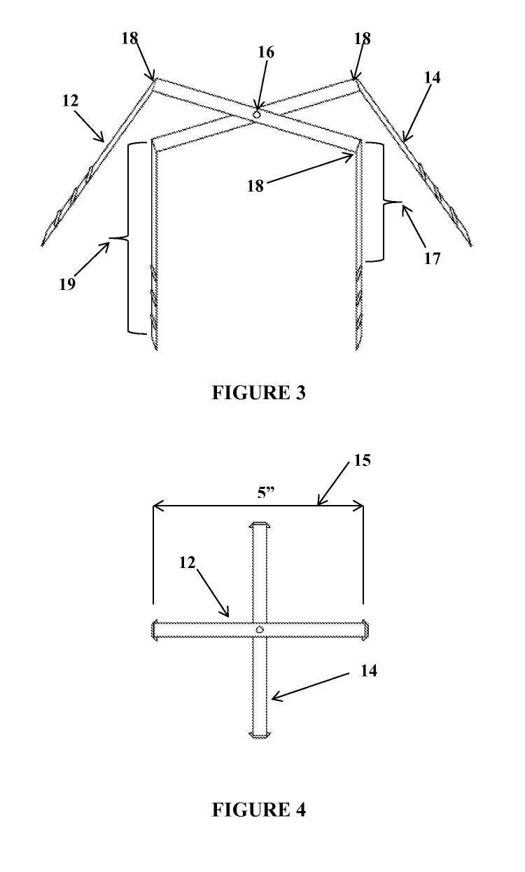

As shown in FIGS. 1 through 4 there are three ridge protrusions located at opposite ends of the first and second member 12 and 14, respectively wherein in one preferred embodiment the ridge protrusion typically 1/16 of an inch. As described before, the first and second members 12 and 14 are both generally elongated and rectangular in shape and may be fabricated from plastic wherein the flexible hinges 18 are defined by small thin sections within the plastic material for allowing portions of the first and said second member to fold back and around thereby forming a protective cage as shown in FIGS. 2 and 3. By way of example but not of limitation, the distance 15 between flexible hinge locations for each member 12 and 14 is approximately 5 inches as shown in FIG. 4. Additionally, by way of example but not of limitation, the distance 17 between flexible hinge to the first ridge point for each member 12 and 14 is approximately 3 inches wherein the distance 19 between flexible hinge to the end point for each member 12 and 14 is approximately 6 inches as shown in FIG. 3.

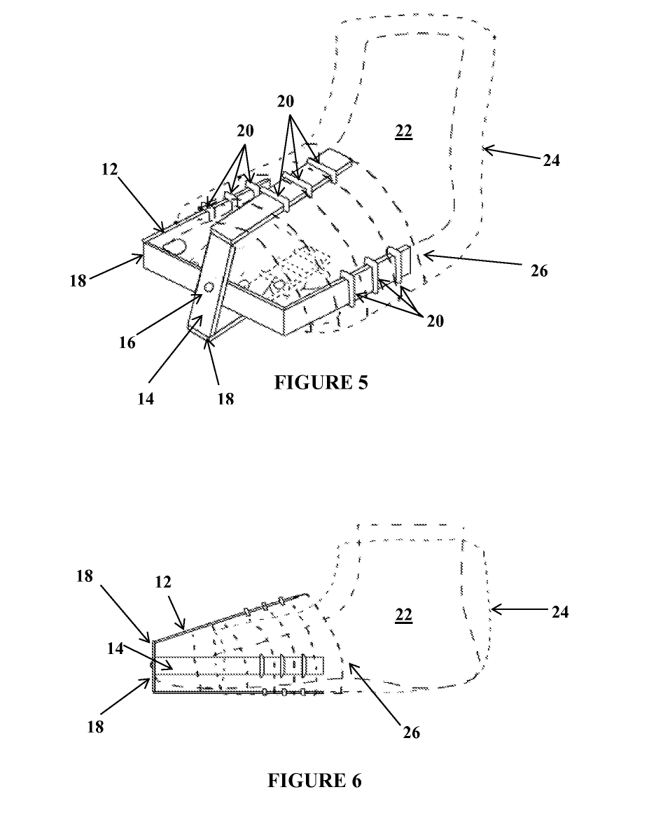

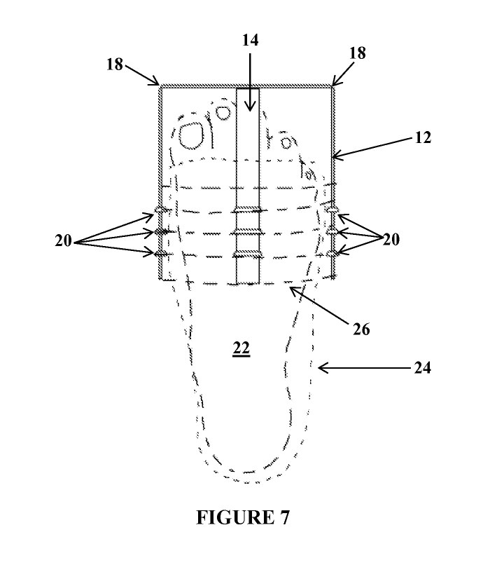

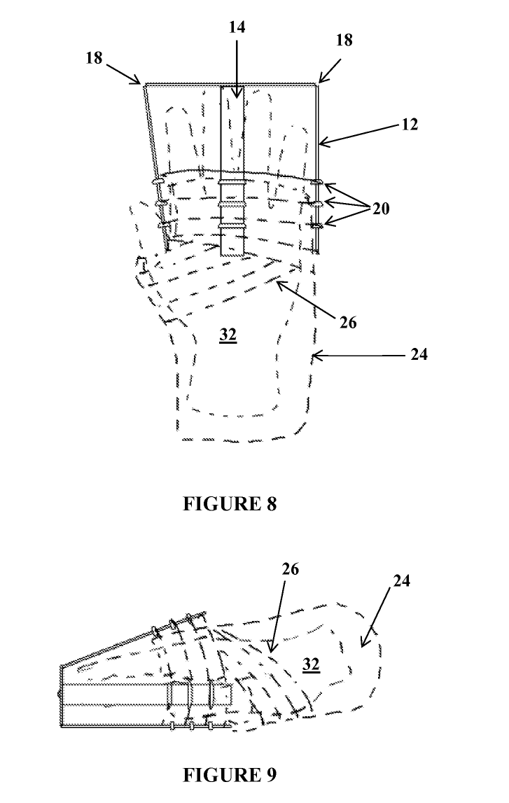

Referring now to FIGS. 5 through 9, in use, a cast or splint is applied in the typical fashion wherein the protective cap 10 of the present invention is bent using flexible hinges 18 and placed over cast 24 in the desired position, and a final roll of cast tape/roll 26 is wrapped around and over the cap 10 using ridge protrusions 20 fixing the cap 10 in place as the cast dries. Once again the cap 10 is made of plastic similar to a zip tie wherein the flexible hinges 18 are simply a small thin section of the plastic where the legs fold down and around the cast 24 allowing members 12 and 14 to form a protective cage. FIGS. 5 through 7 illustrates this use for a foot 22 wherein FIGS. 8 and 9 illustrate the use of the cap 10 with a hand.

Referring once again to FIGS. 5 through 9, more specifically the rotating/swivel point 16 is adjustable when applying the cap 10 to the cast 24 wherein the cap 10 may be placed in any position (90 or 45 degree relationship to each other would be most common). It should be understood that although the figures depict an average (medium) size foot. Small, medium, large, and pediatric would be available. Also, the cap for hand/fingers 32 would be two to three inches longer from hinge 18 to first leg ridge 20 allowing more length for fingers. Turning once again to FIGS. 8 and 9, with a hand 32 one would start the cast 24, place the cap 10 then put a final layer of fiberglass wrap 26 down. That way the cap 10 is trapped between the sticky layers of fiberglass 26 and is held or trapped there as the fiberglass hardens.

In summary, the finger and toe cap is intended for combination with a splint, plaster or fiberglass cast of the type which is applied to the hand or foot of a fracture patient leaving the fingers or toes uncovered and protected. The cap comprises two elongated linear members each defining a plurality of flexible hinges and ridge protrusions along their length wherein the members are connected together for rotation. In use, these members are rotated and partially bent using the hinges for attachment using the ridge protrusions to opposite sides at the end of the cast whereby the cap protects a patient's digital extremities during orthopedic healing by forming a protective cage.

It is contemplated for embodiments of the invention to extend to individual elements and concepts described herein, independently of other concepts, ideas or system, as well as for embodiments to include combinations of elements recited anywhere in this application. Although illustrative embodiments of the invention have been described in detail herein with reference to the accompanying drawings, it is to be understood that the invention is not limited to those precise embodiments. As such, many modifications and variations will be apparent to practitioners skilled in this art. Accordingly, it is intended that the scope of the invention be defined by the following claims and their equivalents. Furthermore, it is contemplated that a particular feature described either individually or as part of an embodiment can be combined with other individually described features, or parts of other embodiments, even if the other features and embodiments make no mention of the particular feature. Therefore, the absence of describing combinations should not preclude the inventor from claiming rights to such combinations.

Although the present invention has been described with reference to specific exemplary embodiments, it will be evident that the various modifications and changes can be made to these embodiments without departing from the broader spirit of the invention. Accordingly, the specification and drawings are to be regarded in an illustrative sense rather than in a restrictive sense.

What is claimed is:

1. A cap for use with a cast comprising:
a first member defining a plurality of flexible hinges and ridge protrusions;
a second member defining a plurality of flexible hinges and ridge protrusions;
said first member and said second member connected together for rotation; wherein said first and said second member are rotated and partially bent using said hinges and attached using said protrusions at opposite ends of the cast whereby the cap protects a patients digital extremities during orthopedic healing.

2. The cap according to claim 1, further comprising protecting patient's toes.

3. The cap according to claim 1, further comprising protecting patient's fingers.

4. The cap according to claim 1, wherein said first member and said second member are connected a center location.

5. The cap according to claim 1, wherein said ridge protrusions are located at opposite ends of said first and second member.

6. The cap according to claim 1, wherein there are three said ridge protrusions are located at opposite ends of said first and second member.

7. The cap according to claim 1, wherein said first and second members are strategically placed over the cast whereby a final roll of cast material is wrapped around and over said first and second members thereby fixing the cap in place as the cast dries.

8. The cap according to claim 1, wherein said flexible hinges are located on each side of said first and second member rotatable connection point at an equal distance.

9. The cap according to claim 1, wherein the cast is made using fiberglass material.

10. The cap according to claim 1, wherein the cast is made using plaster.

11. The cap according to claim 1, also used with splints.

12. The cap according to claim 1, wherein said first and second members are both generally elongated and rectangular in shape and made from plastic wherein said hinges are defined by small thin sections within said plastic material for allowing portions of said first and said second member to fold back and around the cast thereby forming a protective cage.

13. The cap according to claim 1, wherein said first and second members are both generally elongated and cylindrical in shape and made from plastic wherein said hinges are defined by small thin sections within said plastic material for allowing portions of said first and said second member to fold back and around the cast thereby forming a protective cage.

14. The cap according to claim 1, wherein said first and second members are quarter inch diameter.

15. The cap according to claim 1, wherein said flexible hinges are 5 inches apart.

16. The cap according to claim 1, wherein said flexible hinges to an end of said first and second members is six inches in length.

17. The cap according to claim 1, wherein said flexible hinge to said ridge protrusion is a three inches in length.
